(19) United States
(12) Patent Application Publication
(10) Pub. No.: US 2007/0282396 A1
(43) Pub. Date: Dec. 6, 2007

(54) System and method of contra-lateral ear stimulation for preserving neuronal survival and plasticity of the auditory system prior to permanent intra-cochlear implantation

(76) Inventors: Edward H. Overstreet, Valencia, CA (US); Albert A. Maltan, Sistrans (AT); Janusz A. Kuzma, Parker, CO (US)

Correspondence Address: ADVANCED BIONICS CORPORATION, 25129 RYE CANYON ROAD, VALENCIA, CA, 91355, US

(21) Appl. No.: 10/954,321

(22) Filed: Sep. 30, 2004

Related U.S. Application Data
(60) Provisional application No. 60/510,841, filed on Oct. 14, 2003.

(51) Int. Cl. A61N 1/36

(52) U.S. Cl. 607/57

(57) ABSTRACT

An implantable microstimulator, or equivalent neural stimulator, generates a relatively simple signal containing temporally challenging information and delivers such signal to the middle or outer portion of the ear contra-lateral to an ear having a cochlear implant of a bilaterally deafened patient. Such stimulation delivered to the contra lateral ear advantageously increases the survival rate of neurons therein, and further helps maintain or extend the plasticity of the higher auditory pathways of the contra-lateral ear, thereby allowing a cochlear implant to be more effectively used in such ear at a later date. The stimulation provided by the microstimulator, or equivalent simple neural stimulator, need not be continuous, but may be provided only during limited periods of time each day, or only on selected days. Further, such stimulation preserves whatever residual hearing may be left in the contra lateral ear.

The present application claims the benefit of U.S. Provisional Patent Application Ser. No. 60/510,841, filed 14 Oct. 2003, which application is incorporated herein by reference in its entirety.

### FIELD OF THE INVENTION

The present invention relates to cochlear implants, and more particularly to the use of (a) a cochlear implant in one ear of a bilaterally deaf cochlear implant patient, and (b) an implantable micro-stimulator in the other ear of the bilaterally deaf patient. The cochlear implant provides the ear in which it is implanted (the “implant ear”) with the basic ability to perceive sound. The micro-stimulator provides the contra-lateral ear (the “non-implant ear”) with simplified electrical stimulation that preserves neuronal survival and plasticity of the auditory system prior to implantation of a cochlear implant in such ear at a later date.

### BACKGROUND OF THE INVENTION

Most bilaterally deaf cochlear implant candidates receive an implant in only one ear. While unilateral hearing provides the basic ability to perceive sound, it fails to provide important bilateral benefits for listening in background noise and for localizing the source of sound signals.

The benefits of bilateral hearing include: (I) the ability to listen with the ear that has the more favorable signal to noise ratio with respect to the sound signal; (ii) interaural timing differences; (iii) interaural intensity differences; and (iv) spectral cues.

Even more importantly, the side of the brain that is connected to the ear that is not used (the “non-implant” ear) loses plasticity over time. This is a particularly critical detriment for young children who receive a cochlear implant in only one ear, where unilateral implantation can result in an irreversible loss of plasticity in the non-implant ear.

The principle reasons why the vast majority of cochlear implant candidates are implanted unilaterally are as follows: (i) reimbursement is often provided only for a single implant; (ii) there is a fear of losing residual hearing in both ears following cochlear implantation; (iii) current cochlear implant systems require external hardware, which is cumbersome to wear bilaterally; and (iv) especially in young children, cochlear implantation is associated with certain risks that are further increased with bilateral implantation. As a result, most children who receive a cochlear implant system are left un-stimulated in the contra-lateral ear during the most critical period of plasticity.

It is thus seen that there is a need for a system and method that allows bilaterally deaf patients, particularly bilaterally deaf young children, who receive a cochlear implant in one ear to preserve the neuronal survival and plasticity of the auditory system of the other (non-implanted) ear.

### BRIEF SUMMARY OF THE INVENTION

The present invention addresses the above and other needs through the use of an implantable micro-stimulator, or equivalent, that provides a relatively simple signal containing temporally challenging information that is delivered to the middle or outer portion of the contra-lateral (non-implanted) ear. Such stimulation advantageously increases the survival rate of neurons in the contra-lateral ear of the bilaterally deafened individual, and further helps maintain or extend the plasticity of the higher auditory pathways of the contra-lateral ear. The stimulation provided by the micro-stimulator, or equivalent simple neural stimulator, need not be continuous, but may be provided only during limited periods of time each day, or only on selected days.

The present invention describes several embodiments of a system or method for stimulating the ear contra-lateral to the cochlear implant in bilaterally deafened patients who receive unilateral cochlear implantation. Advantageously, the embodiments described require minimal surgical intervention, if any, and may be carried out without the need for intra-cochlear electrodes.

It is a feature of the invention to provide simple neural stimulation of the auditory system of the ear contra-lateral to the cochlear implant in a bilaterally deafened patient in a way that preserves neuronal survival and plasticity of the auditory system of the contra lateral ear.

It is a further feature of the invention to provide such stimulation to the contra-lateral ear in a way that preserves residual hearing.

While intended primarily for bilaterally deafened young children, under special circumstances, the invention may also be used with older children or adults.

In accordance with one embodiment, the invention may be characterized as a method for preserving neuronal survival and plasticity of the auditory system of the ear contra lateral to ear having a cochlear implant of a bilaterally deafened patient. Such method includes: (a) placing an extra-cochlear electrode at a specified location within or on a side of the head of the patient contra lateral to the cochlear implant; and (b) providing a selected regime of electrical stimulation pulses through the extra-cochlear electrode to tissue surrounding the extra-cochlear electrode, which electrical stimulation pulses are adapted to preserve neuronal survival and plasticity of the auditory system of the contra-lateral ear of the patient.

In accordance with another embodiment, the invention may be characterized as a system for preserving neuronal survival and plasticity of the auditory system of an ear contra lateral to an ear having a cochlear implant of a bilaterally deafened patient. Such system includes: (a) an extra-cochlear electrode adapted to be placed at a specified location within or on a. side of the head of the patient contra lateral to the cochlear implant; and (b) an electrical stimulator connected to the extra-cochlear electrode having means for generating a regime of electrical stimulation pulses. The electrical stimulator further has means for controlling when and with what intensity the electrical stimulation pulses are applied to the extra-cochlear electrode. Advantageously, the regime of electrical stimulation pulses, when applied through the extra-cochlear electrode, are adapted to preserve neuronal survival and plasticity of the auditory system of the contra-lateral ear of the patient, thereby allowing a cochlear implant to be more effective if implanted in the contra-lateral ear at a later date.

### BRIEF DESCRIPTION OF THE DRAWINGS

The above and other aspects of the present invention will be more apparent from the following more particular description thereof, presented in conjunction with the following drawings wherein:

FIG. 1 is a cross-sectional side view of a mesh, ball electrode that may be used with one embodiment of the invention;

FIG. 2 is a sectional view of the middle-ear, and illustrates a representative placement of the mesh, ball electrode;

FIG. 3 is a side view of relevant portions of the middle-ear/inner-ear interface, and illustrates a preferred manner of placing the mesh, ball electrode in the niche or recess in front of the round window;

FIG. 4 is a sectional view of relevant portions of the middle-ear and outer-ear, and illustrates a preferred placement of an implantable neurostimulator, such as the BION stimulator, that is electrically connected to the mesh, ball electrode within the middle-ear in accordance with one embodiment of the present invention; and

FIG. 5 is a flow chart that illustrates the basic method of the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

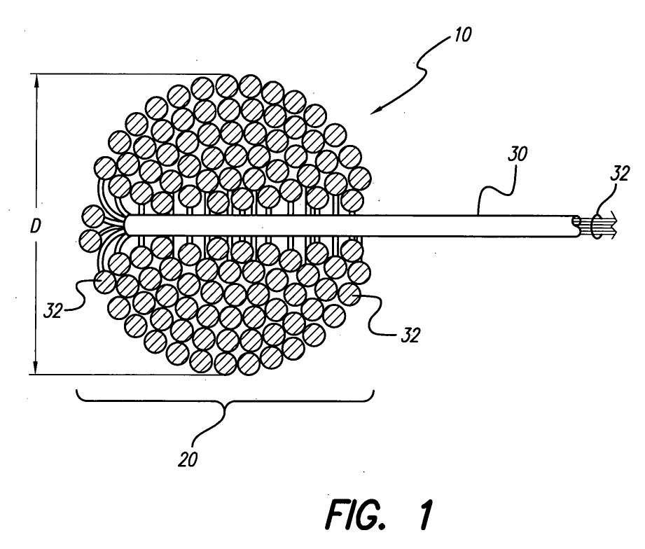

Turning first to FIG. 1, a cross-sectional side view of a mesh, ball electrode is shown. A mesh, ball electrode 10 is made by wrapping the wires of a cable 30 around a suitable mandrel (not shown) to form a ball-shaped head 20 having a diameter “D” from 1.5 to 2.5 mm. The cable 30 is preferably made from an insulated multi-strand cable, having multiple wires or strands 32. In one embodiment, the cable 30 may be made from Teflon-insulated 9- or 11-strand Pt/Ir wires 32. The length of the wires 32 may be about 200 mm, sixty (60) mm of which forms the cable 30, forty (40) mm of which extends out from the cable, e.g., so that the wires can be connected to a suitable pulse generator, and sixty-to-one hundred (60-100) mm of which are used to form the ball-shaped head 20 of the ball electrode 10. Each lead wire 32 is, at a proximal end, welded to platinum pins (not shown) on a neurostimulator, or to a connector that attaches to a neurostimulator, or to a BION-type stimulator, or otherwise electrically connected to a suitable pulse generator.

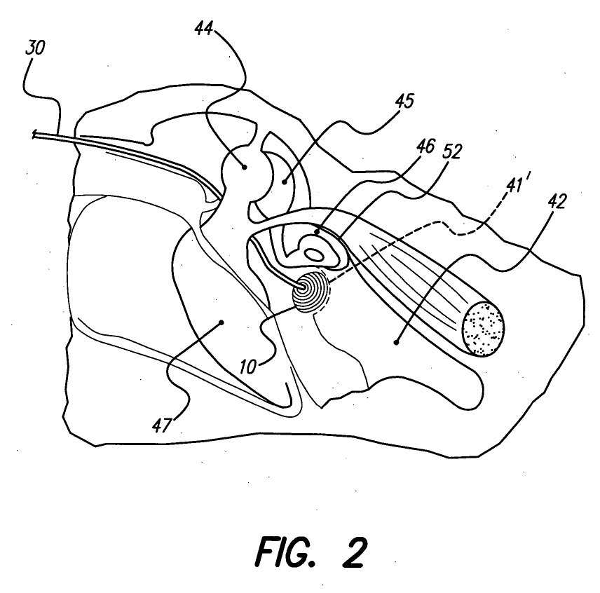

To form the ball-shaped head 20 of the electrode, a sixty-to-one hundred (60-100) mm length of insulated wire 32 is stripped and annealed at a temperature of 1000-1200 C., after which it is allowed to cool at room temperature. Then, the wire is wrapped using a mandrel (not shown), as generally described in FIGS. 2B-2F of U.S. Pat. No. 4,809,712, incorporated herein by reference.

The mandrel has a diameter of about 0.45 mm and a tip having a length of between about 1.5-2.5 mm. A notch having a width of about 0.15 mm is also located at the tip. The notch is placed around the end of the remaining insulation of the cable 30, while the wires or strands 32 are wrapped around the mandrel twenty-five to forty times (depending upon the diameter “D” of the ball that is desired) to form the ball electrode 10 with unfixed turns and an outer diameter “D” of 1.5-2.5 mm. Once the ball-shaped head 20 is formed, the mandrel is pulled gently away from ball electrode 10, leaving the ball-shaped head 20 intact. FIG. 1 illustrates a cross-sectional side view of the ball electrode 10. Note that the electrode ball-shaped head 20 is porous in the sense that the winding process leaves spaces between adjacent turns.

FIG. 2 illustrates the mesh, ball electrode 10 used with the present invention positioned in one preferred location in front of the round window 42 of the contra-lateral ear of the deafened individual. (Note, as used herein, the term “contra-lateral” is an adjective that describes an item or feature on the side of the bilaterally deafened individual's head opposite the side where the cochlear implant is located. Thus, if the cochlear implant is implanted in the individual's right ear, the contra-lateral ear is the left ear, the contra-lateral side of the individual's head or skull is the left side of the head or skull.) For purposes of the description of the mesh, ball electrode 10 presented herein, such mesh, ball electrode 10 is always implanted in or near the contra-lateral ear.

As illustrated in FIG. 2, the cable 30 may re routed through the middle-ear, past the malleus 44, incus 45, and stapes 46, without significantly interfering with their normal operation, thereby preserving residual hearing of the contra-lateral ear. One advantage of the present invention is that such cable 30 may be routed through the middle-ear using standard middle-ear surgical procedures performed under a local anesthesia, behind the skin and along the bone of the ear canal, to the micro-stimulator, or other neuro-stimulator, which is placed under the skin or recessed in the temporal bone, or other suitable location in the contra-lateral side of the skull.

An outline of the normal cavity, niche, or recess, that is located on the middle-ear side of the round window 42 of the contra-lateral ear is depicted by the dotted line 41′. Applicants have discovered that by placing the mesh ball electrode 10 within this cavity, or recess, or in another suitable extra-cochlear location of the ear, and by then applying an electrical stimulus through this electrode, sufficient temporal information is provided to the middle-ear/inner-ear to help maintain or extend the plasticity of the higher auditory pathways of the contra-lateral ear. Moreover, such electrical stimulus preserves residual hearing of the contra-lateral ear. This preservation of the plasticity of the contra-lateral ear makes the subsequent implantation of a cochlear implant therein much more effective and efficient.

Advantageously, placement of the extra-cochlear electrode 10 may be accomplished under local anesthesia, thereby significantly reducing the cost and trauma associated with cochlear implant surgery.

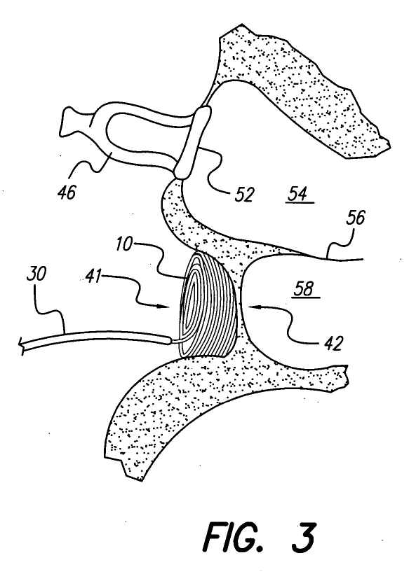

FIG. 3 depicts the middle-ear/inner-ear interface of the contra-lateral ear. The oval window 52 separates the scala vestibuli 54 (one of the three parallel ducts that traverses the spiral-shaped cochlea) from the middle-ear. The stapes 46 attaches to the oval window 52 on the middle-ear side of the oval window. The stapes 46, in turn, is mechanically coupled through the incus 45 and malleus 44 to the ear drum, or tympanic membrane 47, as seen in FIG. 2. Pressure waves (sound waves) sensed through the outer-ear are directed to the tympanic membrane 47 through the ear canal, causing it to vibrate. Such vibrations are then coupled through the malleus 44, incus 45 and stapes 46 of the middle-ear to the oval window 52. Vibrations of the oval window in turn cause vibrations of the fluid within the scala vestibuli 54 of the cochlea. Such fluid vibrations are further coupled through the basilar membrane 56 to the scala tympani 58 (another of the parallel ducts that traverse the cochlea). The oval window 52 thus forms a barrier between the scala vestibule 54 and the middle-ear; and the round window 42 similarly forms a barrier between the scala tympani 58 and the middle-ear. The round window 42 resides in a niche 41, or recess, of the middle ear. This niche 41, or recess, is one preferred extra-cochlear location where the mesh, ball electrode 10 may be placed.

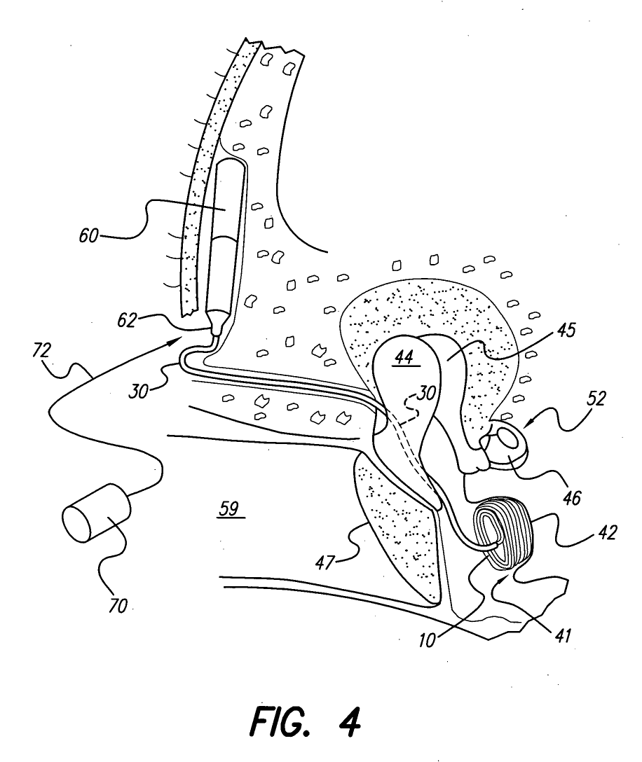

FIG. 4 illustrates a partial side view of outer-ear/middle ear interface of the contra-lateral ear. In a normal-functioning ear, sound waves enter the outer-ear through the ear canal 59 and strike the tympanic membrane (ear drum) 47, causing it to vibrate. Such vibrations are transferred through the three tiny bones of the middle ear, the malleus 44, the incas 45, and stapes 46, to the oval window 52. The interface barrier between the outer-ear and the middle-ear is the tympanic membrane 47. The interface between the middle-ear and the inner-ear comprises the oval window 52 and the round window 42. As previously indicated, the round window 42 resides within a niche, or recess, 41 of the middle-ear. The mesh, ball electrode 10 of the present invention may be placed within the niche or recess 41.

FIG. 4 also shows a preferred placement of an electrical stimulator 60, e.g., a BION® microstimulator device, manufactured by Advanced Bionics Corporation of Valencia, Calif. A BION stimulator 60 is a single channel leadless stimulator, but for purposes of the present invention, may have the cable lead 30 connected thereto by way of a slip-on or snap-on connector 62, or equivalent. The BION stimulator 60 is described more fully, e.g., in U.S. Publication No. US 2004/0059392A1, which publication is assigned to the same assignee as is the present application, and is incorporated herein by reference. A representative connector 62 that may be used to add a lead to such a BION-type stimulator is disclosed in International Publication Number WO 03/063951 A1, published Aug. 7, 2003, (International Application Number PCT/US03/02784), also incorporated herein by reference.

As described in the referenced documents, one preferred embodiment of a BION microstimulator includes its own rechargeable power source, i.e., a rechargeable battery. Other BION microstimulators may receive operating power through a close-field RF field. Either type of microstimulator—powered from a self-contained rechargeable power source or from a close-field RF field.—may be used with the invention.

A microphone 70 may be coupled to the stimulator 60 by way of a signal communication link 72. A preferred location for the microphone 70 is in the canal of the contra-lateral ear. A preferred link 72 for linking the microphone 70 to the stimulator 60 is a wireless radio frequency (RF) link. However, other suitable links may be used, such as a wire link.

The microphone 70 also preferably includes processing circuitry to process and condition the signal that is sent to the stimulator 60 over the link 72. Such processing circuitry detects the sound or acoustic signals sensed by the microphone's transducer, converts them to electrical signals, amplifies the electrical signals, and processes the amplified electrical signals to determine if they represent an appropriate signal that should trigger the BION stimulator 60 so as to cause it to generate an electrical stimulation pulse that is sent to the mesh, ball electrode 10. Such processing, in one embodiment, involves amplifying and filtering the electrical signal received from the microphone's transducer, and determining the derivative thereof, which derivative signal may then be used as a trigger signal for the BION stimulator 60 only when the amplified and filtered and processed signal meets prescribed criteria.

In accordance with one embodiment of the invention, the mesh, ball electrode 10 is placed in the recess on the middle ear side of the round window 42, or at or in some other suitable extra-cochlear location within the middle-ear, of the user's deaf ear. The cable 30 is routed and connected to the stimulator 60. The stimulator 60 is then coupled to the microphone 70, or other external programming device, so as to cause the stimulator 60 to generate appropriate stimuli that provides temporal information to the ear. The stimuli pattern, or regime, may vary from patient to patient, but will typically involve applying mono-polar biphasic stimulus currents to the tissue surrounding the electrode at a relatively low current level, e.g., less than 1 or 2 ma peak in accordance with a prescribed regime, as described below. Typically, a return electrode will be located on the case of the stimulator 60, but it may also be placed in other suitable locations by way of an additional lead or cable connected to the stimulator, or an additional electrode placed on the cable 30 (but having it's own separate electrical connection).

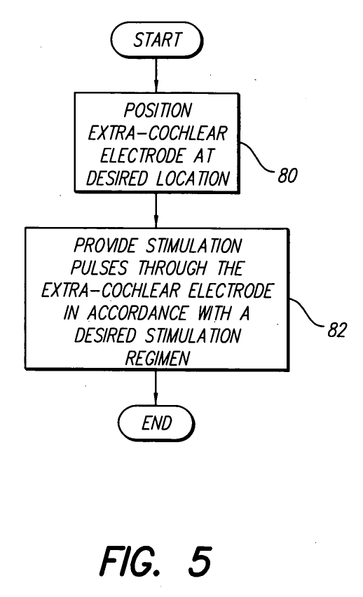

FIG. 5 is a flow chart that illustrates the basic method of the invention. As seen in FIG. 5, the method involves two fundamental steps, or procedures. First, as seen in block 80, an extra-cochlear electrode is placed at or in a desired location on the contra-lateral side of the individual's head, e.g., in the contra-lateral ear. The extra-cochlear electrode may be a mesh, ball electrode 10 as described previously, or may be any other type of electrode suitable for the location where it is placed. The desired location where the electrode is placed may be any suitable location within the middle-ear or outer-ear, or even on the skin surface of the patient. For example, the electrode may be placed in the round window niche or on the promontory in the middle-ear through a standard middle-ear surgical procedure. Alternatively, the electrode may be placed temporarily in the ear canal. Still alternatively, the electrode may be a surface or TENS type electrode that is placed somewhere on the skin on the skull.

Still with reference to FIG. 5, it is seen that the second step or procedure associated with the method of the invention involves providing stimulation pulses through the extra-cochlear electrode in accordance with a desired stimulation pattern or regime (block 82). The stimulation pattern or regime may take many forms, as may the source of the stimulation pattern or regime. For example, in one embodiment, stimulation may comprise a simple pulse train. In other embodiments, the stimulation may comprise pulse trains with variable duty cycles or frequency, or waveforms that contain temporally challenging information, all of which may be turned on during selected periods of time. The stimulator 60, or source of the stimulation pulses, may comprise a BION microstimulator, as described in the referenced documents, or other suitable pulse generator.

In still further embodiments, the stimulation pulses may comprise a real-time derivative of a sound signal that is recorded or sensed through a microphone, e.g., the microphone 70, from the environment of the patient. Thus, in such embodiment, the stimulator may comprise a modified hearing aid.

In yet additional embodiments, the stimulation pulses may be either a processed version of an acoustic input that is sensed or recorded through a microphone, or a random signal. In other embodiments, the stimulation pulses or stimulation waveform may comprise a pre-recorded or stored signal that has properties of a sound signal.

As described above, it is thus seen that the present invention involves the use and placement of an extra-cochlear electrode connected to a microstimulator, such as a BION microstimulator, or other suitable stimulator or source of stimulation signals. The stimulation signals are applied to the extra-cochlear electrode placed on or in the contra-lateral ear in accordance with a desired stimulation pattern or regime, which pattern or regime is selected to preserve residual hearing, and to help maintain or extend the plasticity of the higher auditory pathways.

The microstimulator, or other small stimulator, used with the invention may be placed under the skin or recessed in the temporal bone or some other location in the skull.

In one preferred embodiment, the microstimulator contains its own power supply, e.g., a rechargeable power supply, or may receive operating power from an external power source through close-field RF coupling.

The microstimulator has the ability to generate a stimulation signal derived from the acoustic input collected from the environment. Such stimulation signal is applied through the extra-cochlear electrode in order to apply electrical stimulation to the location where the electrode is positioned.

A microphone, or similar transducer, may be used to collect or record acoustic input from the environment. This acoustic input may then be processed, e.g., through a suitable filter or other circuitry, that determines the derivative of the acoustic input. The resulting derivative signal may then be applied to the extra-cochlear electrode, or further processed, e.g. by a microstimulator, so that when the acoustic input or derivative thereof meets certain prescribed criteria, e.g., exceeds a prescribed intensity threshold, or has frequency components above a certain intensity within a prescribed frequency band, the microstimulator generates a stimulation pulse that is applied to the extra-cochlear electrode. The microphone may be worn externally to the stimulator and interface with the. stimulator via a wireless radio frequency (RF) link. Alternatively, the microphone may be connected to the simulator via a wired link.

One embodiment of the invention includes an extra-cochlear stimulating electrode, such as a mesh ball electrode described in U.S. Patent Application Ser. No. 10/932,812, filed Sep. 1, 2004, incorporated herein by reference. As disclosed in pending application Ser. No. 10/932,812, one preferred embodiment comprises a mesh ball electrode having a doughnut shape. Another preferred embodiment in the same pending application comprises a mesh ball electrode made from a multi-strand wires having a zig-zag pattern and also forming a doughnut shape ball electrode. Such mesh ball electrode 10, or other similar electrode, may be placed in the round window niche or on the promontory, or in some other extra-cochlear location. Such an electrode can be placed through a standard middle-ear surgical. procedure, and a thin cable can be routed behind the skin and along the bone of the ear canal. The second end of the cable may then be connected to an implantable microstimulator, or other suitable source of stimulation pulses.

Stimulation provided to the electrode can be simple pulse trains, pulse trains with variable duty cycles or frequency, or waveforms that contain temporally challenging information. Stimulation may also be turned on during selected periods of time, and turned off at other periods of time.

The microstimulator may be a BION® type microstimulator, or a similar device. Such device may be placed in the skull or recessed in the temporal bone or some other part of the skull.

Further, the microstimulator may be an RF driven device, i.e. a device that receives its operating power and/or stimulus information via an RF transmitter. The RF transmitter includes an external coil that can be integrated into a pillow, a stroller, or some other place that is in relatively close proximity to the head of a small child during some periods of time during the day.

Alternatively, the microstimulator may contain its own battery, either a primary battery or a rechargeable battery.

It is further noted that the microstimulator may be programmable, which means that it may be adjusted to provide stimulation at different stimulus amplitudes or levels. Programmability further means that stimulation levels, or other stimulation parameters, may be adjusted with the assistance of data from objective measurements, such. as auditory brainstem potentials, mid latency potentials, and the like.

In another preferred embodiment, the microstimulator used with the extra-cochlear electrode generates a signal that comprises a real-time derivative of a sound signal that is recorded from the environment, e.g., through the use of a microphone. The microphone that records the sound signal may be part of the microstimulator or may be worn separately and interface with the micro-stimulator via a wireless link, such as an RF link. The signal generated by the microstimulator may be a single channel broadband signal that covers part or all of the frequency range of speech or sound. Alternatively, the signal generated by the microstimulator may contain only one or several features extracted from the sound or speech recorded from the environment.

In yet another embodiment of the invention, a stimulating electrode is placed on the round window 42 or on the promontory, as described previously, and an electrical path to the electrode is provided by a conductor. The conductor may be similar in concept to a middle-ear tube, i.e. the conductor may protrude through the tympanic membrane and terminates in the ear canal. A stimulator, such as a modified hearing aid, may then be connected to the electrode via the conductor during times when it is desired to provide stimulation. Such stimulator, like a hearing aid, may be programmable and may be adjusted to provide stimulation at different stimulus amplitudes or levels. Stimulation levels, or other stimulation parameters, may be adjusted with the assistance of data from objective measurements, such as auditory brainstem potentials, mid latency potentials, or the like.

In an additional embodiment, a stimulating electrode is temporarily placed in the ear canal. Such electrode may be mounted on a shell, such as an In-The-Canal (ITC) housing. The electrode is then connected to a stimulator that is similar to a hearing aid and which generates an electrical signal. The signal may either be a processed version of an acoustic input that is recorded via a microphone, or a random signal. The stimulator is preferably programmable and may be adjusted to provide stimulation at different stimulus amplitudes or levels. Stimulation levels, or other stimulation parameters, may be adjusted with the assistance of data from objective measurements, such as auditory brainstem potentials, mid latency potentials, or the like.

In still another embodiment, stimulation is provided via a surface (or TENS type) electrode that is placed somewhere on the skin on the skull. The surface electrode is connected to a stimulator that may be of any shape or kind, and which may be connected to the electrode via a cable or other connecting method. The stimulation waveform applied through the surface electrode may be a derivative of sound that is collected from the environment via a microphone. Alternatively, the stimulation waveform may be a pre-recorded or stored signal that has properties of a sound signal, i.e. constant variation in content. The stimulation waveform may also be a random signal that contains temporally challenging information, or other variability. The stimulator may be programmable and may be adjusted to provide stimulation at different stimulus amplitudes or levels. Additionally, stimulation levels, or other stimulation parameters, may be adjusted with the assistance of data from objective measurements, such as auditory brainstem potentials, mid latency potentials, or the like.

It is noted that the above-described embodiments may be applied to the contra-lateral ear of a patient who receives a cochlear implant in one ear. That is, cochlear implantation of the second ear may not be possible due to financial or reimbursement reasons, while the concepts described above are simpler and thus less expensive and more affordable. Intra-cochlear implantation of the second ear may be postponed because the patient or clinician may want to preserve one ear for later cochlear implant technology. In such cases, the concepts described above can be applied in an effort to preserve auditory plasticity in the non-implanted ear.

While the invention herein disclosed has been described by means of specific embodiments and applications thereof, numerous modifications and variations could be made thereto by those skilled in the art without departing from the scope of the invention set forth in the claims.

What is claimed is:

1. A system for preserving neuronal survival and plasticity of the auditory system of an ear contra lateral to an ear having a cochlear implant of a bilaterally deafened patient, comprising:
an extra-cochlear electrode adapted to be placed at a specified location within or on a side of the head of the patient contra lateral to the cochlear implant; and
an electrical stimulator connected to the extra-cochlear electrode having means for generating a regime of electrical stimulation pulses, the electrical stimulator having means for controlling when and with what intensity the electrical stimulation pulses are applied to the extra-cochlear electrode;
wherein the regime of electrical stimulation pulses, when applied through the extra-cochlear electrode, are adapted to preserve neuronal survival and plasticity of the auditory system of the contra-lateral ear of the patient.

2. The system of claim 1 wherein the extra-cochlear electrode comprises a mesh, ball electrode adapted to be placed within the contra-lateral middle ear of the patient.

3. The system of claim 2 wherein the mesh, ball electrode is adapted to be placed in or near the round window niche of the contra-lateral middle ear of the patient.

4. The system of claim 1 wherein the extra-cochlear electrode comprises an electrode adapted to be placed within the canal of the contra-lateral ear of the patient.

5. The system of claim 1 wherein the extra-cochlear electrode comprises a surface electrode adapted to be placed on the contra-lateral side of the skull of the patient.

6. The system of claim 1 wherein the system is adapted to be used in a very young patient of less than two years old.

7. The system of claim 1 wherein the system is adapted to be used in a patient of more than two years old with a hearing impairment.

8. The system of claim 1 wherein the regime of electrical stimulation pulses that are applied through the extra-cochlear electrode are selected from the group of stimulation pulses comprising simple pulse trains, pulse trains with variable duty cycles or frequency, and waveforms containing temporally challenging information.

9. The system of claim 1 wherein the electrical stimulator comprises an implantable microstimulator.

10. The system of claim 9 wherein the implantable microstimulator includes a connector and an insulated multi-strand cable having multiple wires connected to the microstimulator through the connector, and wherein the multi-strand cable has an electrode at a distal end thereof.

11. The system of claim 10 wherein the insulated multi-strand cable comprises a ball-shaped electrode.

12. The system of claim 9 wherein the implantable microstimulator comprises a BION® type microstimulator.

13. The system of claim 9 wherein the implantable microstimulator comprises a pulse generator electrically connected to a mesh ball electrode.

14. The system of claim 13 wherein the mesh ball electrode comprises a ball electrode having a diameter of 1.5 to 2.5 mm.

15. The system of claim 9 wherein the implantable microstimulator comprises a radio-frequency (rf) driven device.

16. The system of claim 9 wherein the implantable microstimulator includes a battery that provides operating power for the micro-stimulator.

17. The system of claim 1 wherein the electrical stimulator comprises a modified hearing aid device.

18. The system of claim 17 wherein the modified hearing aid device includes means for sensing a sound signal from the environment of the patient, and means for generating the stimulation signal as a signal derived from the sensed sound signal.

19. The system of claim 18 wherein the signal derived from the sensed sound signal comprises a real-time derivative of the sensed sound signal.

20. A method for preserving neuronal survival and plasticity of the auditory system of an ear contra lateral to an ear having a cochlear implant of a bilaterally deafened patient, comprising:
placing an extra-cochlear electrode at a specified location within or on a contra lateral side of the head of the patient, opposite the side of the head of the patient having the cochlea implant; and
providing a selected regime of electrical stimulation pulses through the extra-cochlear electrode to tissue surrounding the extra-cochlear electrode, which electrical stimulation pulses are adapted to preserve neuronal survival and plasticity of the contra-lateral ear of the patient.

21. The method of claim 20 wherein the extra-cochlear electrode comprises a mesh ball electrode.

22. The method of claim 21 wherein the mesh ball electrode comprises a ball electrode having a diameter of 1.5 to 2.5 mm.

23. The method of claim 20 wherein placing the extra-cochlear electrode comprises placing the extra-cochlear electrode in or near the round window niche or promontory of the contra-lateral middle ear of the patient.

24. The method of claim 20 wherein placing the extra-cochlear electrode comprises placing the extra-cochlear electrode within the canal of the contra-lateral ear of the patient.

25. The method of claim 20 wherein placing the extra-cochlear electrode comprises placing the extra-cochlear electrode on a contra-lateral side of the skull of the patient.

26. The method of claim 20 wherein providing a selected regime of electrical stimulation pulses comprises selecting and generating a regime of electrical stimulation pulses from the group of stimulation pulses comprising simple pulse trains, pulse trains with variable duty cycles or frequency, and waveforms containing temporally challenging information.

27. The method of claim 20 further including sensing a sound signal from the environment of the patient, and generating the stimulation signal to be a signal derived from the sensed sound signal.

28. The method of claim 27 wherein generating the stimulation signal comprises generating the stimulation signal to be a real-time derivative of the sensed sound signal.
